(19) United States
(12) Patent Application Publication
(10) Pub. No.: US 2011/0106219 A1
(43) Pub. Date: May 5, 2011

(54) SHORT-PULSE NEURAL STIMULATION SYSTEMS, DEVICES AND METHODS

(76) Inventors: Lawrence J Cauller, Plano, TX (US); Scott Armstrong, Austin, TX (US)

(21) Appl. No.: 12/611,105

(22) Filed: Nov. 2, 2009

(51) Int. Cl. A61N 1/36

(52) U.S. Cl. 607/72; 607/2

(57) ABSTRACT

Methods, devices and systems for neural stimulation using a short-pulse stimulation are described. Using a waveform that generates a sufficiently large capacitive current density in the tissue surrounding a nerve allows neural stimulation at one hundredth the power of a charge injection stimulation. A capacitive discharge may be used to generate the short-pulse stimulation waveform. Short pulse stimulation may be used to generate parasthesia, particularly for treatment of chronic pain.

### CROSS REFERENCE TO RELATED APPLICATIONS

This application is related to co-pending application, Ser. No. ______ filed ______ and entitled “Parasthesia Using Short-Pulse Neural Stimulation Systems, Devices And Methods” (Attorney Docket MTI-056) and to Ser. No. 12/323,854 filed Nov. 26, 2008, entitled “Implantable Transponder Systems and Methods”, all of which are incorporated by reference in their entirety.

### BACKGROUND

The invention relates to the field of neural stimulation, in particular to implanted extra-neural electrical stimulation systems.

Extra-neural electrical stimulation is the application of electrical energy in the tissue near a nerve, resulting in an action potential in the nerve. One well-known method of stimulating a nerve is the charge injection stimulation method, where a constant-current stimulation is generated between implanted electrodes, near the nerve. By applying a constant-current stimulation over a duration, charge is injected into the tissue, where the charge delivered equals the current times the duration. For every given nerve, there is a minimum charge that must be injected into nearby tissue to generate an action potential within the particular nerve. For example, an A-type nerve fiber may be stimulated by the delivery of about 30-40 nanocoloumbs (nC) within a duration of about 100-250 microseconds (μs).

The charge-injection charge delivery threshold represents the minimum charge that must be delivered to stimulate a given nerve by the charge-injection method. The charge delivery threshold for a given nerve can be determined by providing a constant current stimulation pulse in proximity to the nerve and measuring the minimum duration necessary to effect stimulation. To measure the charge delivery threshold for a given nerve, a 2 mA constant current stimulation is delivered between platinum electrodes. The electrodes are positioned at a distance less than 2 millimeters from the exterior membrane of the axon but external to the axon. Each electrode has a surface area of sixteen square millimeters. The minimum duration measured for stimulation of the target nerve, with the current, defines the charge-injection charge delivery threshold for the nerve. It is recognized that some nerves may require variation of the given parameters to effectively stimulate the nerve, however, it will be apparent to those having ordinary skill in the art that the charge-injection mechanism will define the charge-injection charge delivery threshold for the target nerve.

With reference to FIGS. 2a, 2b and 2c, graphs depict three typical stimulation pulses. FIG. 2a is a graph of a stimulation pulse having a constant current of about 0.4 milliamps (mA) and a pulse duration of 100 μs. The charge delivered is equal to the area under the graph, in this case about 40 nC. FIG. 2b is a graph of a stimulation pulse with an exponential waveform, where an initial current of about 0.6 mA reduces to about 0.4 mA in a pulse having a pulse duration of about 100 us. The charge delivered is about 50 nC. FIG. 2c is a graph of a stimulation pulse resulting from three sequential exponential pulses forming a stimulation pulse having a pulse duration of about 100 μs. Each of the exponential pulses are insufficient to stimulate the nerve but the sequence of exponential pulses injects, in sum, a sufficient total charge. The charge delivered is about 75 nC. These pulses are described as examples of charge injection pulses.

With charge injection stimulation, for any given nerve, there is a threshold amount of charge that must be delivered to the tissue near the nerve to effect stimulation.

### SUMMARY

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIG. 1 is a diagram depicting a neural stimulation system in accordance with an embodiment;

FIG. 2 is a series of graphs depicting stimulation pulses;

FIG. 3 is a circuit diagram depicting a stimulation circuit in accordance with an embodiment;

FIG. 4 is a circuit diagram depicting a stimulation circuit in accordance with an embodiment;

FIG. 5 is a block diagram depicting a neural stimulation system, in accordance with an embodiment; and

FIG. 6 is a block diagram depicting a neural stimulation system, in accordance with an embodiment; and

FIG. 7 is a diagram depicting a short-pulse neural stimulation system to generate parasthesia, in accordance with an embodiment.

### DETAILED DESCRIPTION OF THE DRAWINGS

The numerous innovative teachings of the present application will be described with particular reference to presently preferred embodiments (by way of example, and not of limitation). The present application describes several inventions, and none of the statements below should be taken as limiting the claims generally. Where block diagrams have been used to illustrate the invention, it should be recognized that the physical location where described functions are performed are not necessarily represented by the blocks. Part of a function may be performed in one location while another part of the same function is performed at a distinct location. Multiple functions may be performed at the same location.

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of .+−.20% or. +−.10%, more preferably .+−.5%, even more preferably. +−.1%, and still more preferably .+−. 0.1% from the specified amount, as such variations are appropriate to perform the disclosed method.

As used herein, the term “electrode” means an electric conductor through which a voltage potential can be measured. An electrode can also be a collector and/or emitter of an electric current. In one embodiment, an electrode is a solid and comprises a conducting metal. Representative conducting metals include noble metals, alloys and particularly stainless steel, platinum, platinum iridium and tungsten. An electrode can also be a microwire, or the term “electrode” can describe a collection of microwires. In one embodiment, electrodes comprise polytetrafluoroethylene (PTFE), coated stainless steel or tungsten microwires. A conductive polymer such as poly(3,4-ethylenedioxythiophene (PEDOT) may be a suitable electrode material.

Stimulation devices may be coated with, or otherwise incorporate, Polyethylene Glycols (PEG), SU-8, Polyethylene Terephthelate (PET), Polyether Urethan (PEU), Polydimethyl Siloxane (PDMS), Collagen, Polyamides, Polycarbonates, Polystyrene, Poly(vinhl alcohol), PEDIOT, or any other suitable material.

As used herein, the term “integrated circuit” refers to a small-scale, electronic device densely packaged with more than one integrated, electrical component. The components are manufactured on the surface of semiconductor material. There are various scales of integrated circuits that are classified based on the number of components per surface area of the semiconductor material, including small-scale integration (SSI), medium-scale integration (MSI), large-scale integration (LSI), very large-scale integration (VLSI), ultra large-scale integration (VLSI).

As used herein, the terms “operator,” “patient” and “subject” are used interchangeably and mean any individual monitoring or employing the present invention, or an element thereof. Operators can be, for example, researchers gathering data from an individual, an individual who determines the parameters of operation of the present invention or the individual in or on which a stimulator array is disposed. Broadly, then, an “operator,” “patient” or “subject” is one who is employing the present invention for any purpose. As used herein, the terms “operator,” “patient” and “subject” need not refer exclusively to human beings, but rather the terms encompass all organisms having neural tissue, such as monkeys, dogs, cats, rodents, etc.

After selecting the nerve or nerves on which the electrodes will be implanted, it is necessary to determine the site at which the electrodes should be placed for initiating the stimulation signal. Any of a variety of nerves may be stimulated, including the peripheral nerves, the vagus, trigeminal, glossopharyngeal, occipital, sciatic, median, sympathetic nerves and any other appropriate nerve or neural tissue.

It should be noted that although the terms “stimulus,” “stimulation,” “stimulation pulse,” electrical stimulus” and the like are used herein to describe the electrical signal by which the desired therapy or therapeutic regimen is delivered to the selected nerves, the response is perhaps better understood to be a modulation of the electrical activity of the nerves.

The waveform of an electrical stimulus is characterized by the change in voltage between a pair of electrodes over time. The duration of an electrical stimulus is defined as the elapsed time between the application of the stimulus until the voltage between the pair of electrodes, having reached a peak voltage, reduces to half the peak voltage. For a constant voltage pulse, the pulse duration will be the full duration of the pulse. For an individual exponential pulse, the pulse duration may be about one-third the duration of the full discharge, depending on the time constant of the pulse.

Stimulation of nerves has been used to treat a variety conditions, particularly conditions which may be ameliorated directly by stimulation of a nerve. Such nerves and conditions include, but are not limited to multiple small peripheral nerves for treatment of arthritis pain; deep brain/cortical stimulation for treatment of one or more of essential tremor, Parkinson's disease, dystonia, depression, tinnitus, epilepsy, stroke pain, and obsessive compulsive disorder; sacral nerve stimulation for the treatment of incontinence, pelvic pain and sexual dysfunction; vagus nerve stimulation for treatment of epilepsy, depression and pathoplastic conditions such as tinnitus, PTSD, stroke; peripheral nerve stimulation for treatment of chronic pain; spinal cord stimulation for treatment of one or more of chronic pain, angina pain, and peripheral vascular disease pain; cochlear nerve stimulation for treatment of profound deafness; pulmonary nerve stimulation for treatment of respiratory support; gastric nerve stimulation for treatment of one or more of obesity, gastroparesis, and irritable bowel syndrome; and occipital nerve stimulation for treatment of headaches/migraine and/or traumatic brain injury.

Nerves may include peripheral nerves, deep brain/cortical nerves, sacral nerve, vagus nerve, spinal cord, cochlear nerve, pulmonary nerve, gastric nerve and occipital nerve.

Neural stimulation may be used to treat neuropathic, arthritic, osteoarthitic, migraine, diabetic neuropathy, fibromyalgia, cancer, AIDS, traumatic brain injury and other related pain indications. Stimulation of hypoglossal nerve may be used in treatment of obstructive sleep apnea. Desynchronization may be induced by stimulation of the vagus or trigeminal nerve as treatment for epilepsy or Parkinson's disease. Stimulation of the pudental nerve may be used in treatment for bladder control. Stimulation may be used to treat pelvic pain in cases of female sexual dysfunction. The Spheno-Palatine Ganglion may be stimulated to increase blood flow to the central nervous system and to increase permeability, allowing drugs to move through the blood brain barrier. Stimulation of the peroneal or sciatic nerves may treat foot drop. Stimulation near hair follicles may be used to treat hair loss. Stimulation of the vagus nerve may be used to treat immune disorders and some psychiatric disorders, such as depression. The auditory nerve may be stimulated in treatment of hearing disorders. The mandibular nerve may be stimulated to effect a lifetime anesthesia for dental treatment. Stimulation of the heart may be used for cardiac pacemaking. The vestibular nerve may be stimulated for balance disorders. Baroreceptors may be stimulated to control blood pressure. The renal nerve may be stimulated for heart failure, hypertension and renal failure. Stimulation of phrenic nerve may treat lung failure that may result from amyotrophic lateral sclerosis, paralysis and other conditions. Stimulation pulses may be introduced in electro-acupuncture, particularly in ear acupuncture. The median nerve may be stimulated for the relief of refractory carpal tunnel syndrome as well as nausea. Stimulation of peripheral nerves may treat deafferentation pain.

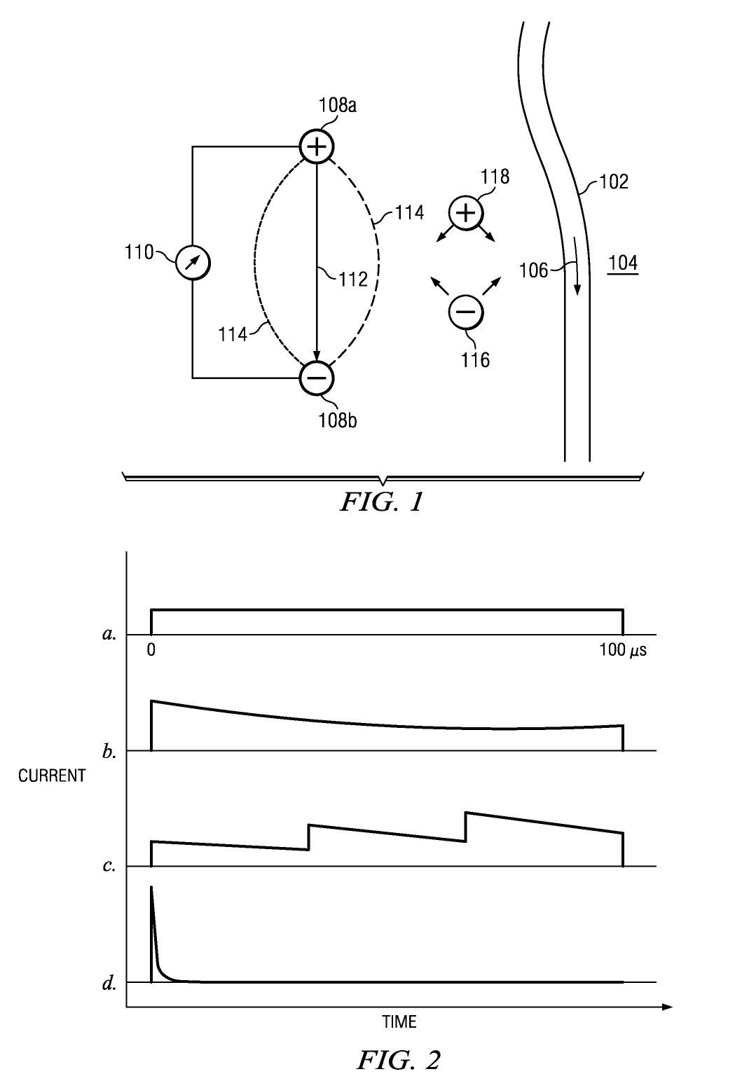

With reference to FIG. 1, a block diagram depicts a neural stimulation system, in accordance with an embodiment. Nerve tissue 102 may be the axon of a nerve. An electrode pair 108, designated individually as 108a and 108b, is placed in near proximity to the nerve 102, within surrounding tissue 104. The electrodes 102a, 102b are electrically connected to a stimulus source 110. Stimulus source 110 may be a charged capacitor, a battery, or any other source of voltage or current. A stimulus is applied to the electrodes 108a, 108b by the stimulus source 110, generating a current 112 between the electrodes 108. The current 112 generates an electric field 114. The electric field 114 attracts and repels electrons 116 and ionized atoms 118 within the tissue 104, generating capacitive currents. These capacitive currents may directly or indirectly cause an action potential 106 in the nerve 102. Similarly, the capacitive currents may directly or indirectly inhibit an action potential 106 in the nerve 102.

With reference to FIG. 2d, a graph depicts a stimulation waveform, in accordance with an embodiment. The stimulation waveform shown is an exponential pulse with a peak current of about 4.5 mA and a pulse duration of about 1 μs. The charge delivered is about 4.5 nC. A pulse of this nature may be characterized by a short duration, less than 50 μs. The pulse may be characterized by the below-threshold charge delivery, less than 25 nC for an Alpha fiber.

Capacitive currents play an instrumental role in short-pulse stimulation. Capacitive currents are proportional to the change in voltage relative to time. A voltage dissipation rate of at least 0.25 V/μs generates the necessary capacitive currents. In accordance with an embodiment, a voltage dissipation rate of 4 V/μs has been effective.

Charge density plays an instrumental role in short-pulse stimulation. The size of the electrodes must be relatively small, less than three square millimeters, to generate the necessary charge density. Another characteristic of a short-pulse stimulation is the absence of low-frequency components in the pulse. For example, an effective short-pulse stimulation may have frequency components greater than 6000 Hz.

Small-pulse stimulation of a peripheral nerve generates the same cortical response generated by charge injection stimulation of the same nerve. Small-pulse stimulation may be effectively used in any neural stimulation treatment that has used charge injection stimulation.

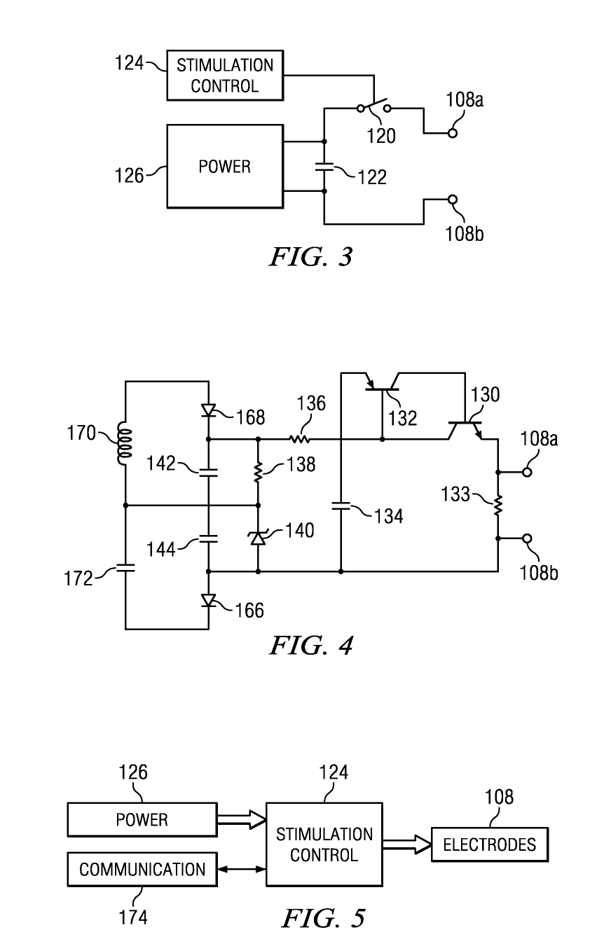

With reference to FIG. 3, a circuit diagram depicts a neural stimulation circuit in accordance with an embodiment. A switch 120 closes and connects a stimulus capacitance 122 between electrodes 108a, 108b to generate a stimulus. The stimulus waveform, in this embodiment, takes the form of exponential decay as the charge stored on the stimulus capacitance 110 is discharged between the electrodes 108a, 108b, generating electric fields in the nearby tissue 104. Stimulation control 124 operates the switch 120 and provides power to the stimulus capacitance 122.

With reference to FIG. 4, a circuit diagram depicts a wireless neural stimulation circuit in accordance with an embodiment. An inductance 170 resonates with resonance capacitance 172 in response to near-field transmissions and generates an oscillating voltage. The diodes 166 and 168 rectify the voltages. Capacitances 142 and 144 sum the rectified voltages. A Zener diode detects a null signal resulting from the near-field transmission and triggers the stimulation pulse. Resistances 138 and 136 affect the voltage levels. The stimulation energy is stored on stimulation capacitor 134. Switches 132 and 132 trigger and latch the stimulation pulse, allowing the stimulation capacitor 134 to discharge between electrodes 108a and 108b. A resistance 133 of about 100 kiloOhms between the electrodes 108a and 108b depolarize the electrodes between stimulation pulses.

With reference to FIG. 5, a block diagram depicts a neural stimulation system in accordance with an embodiment. A stimulation pulse is delivered between electrodes 108. Stimulation control 124 provides the stimulation pulse in accordance with stimulation parameters. The stimulation control 124 receives power from a power source 126. The stimulation parameters may be provided to the stimulation control by a communication system 174.

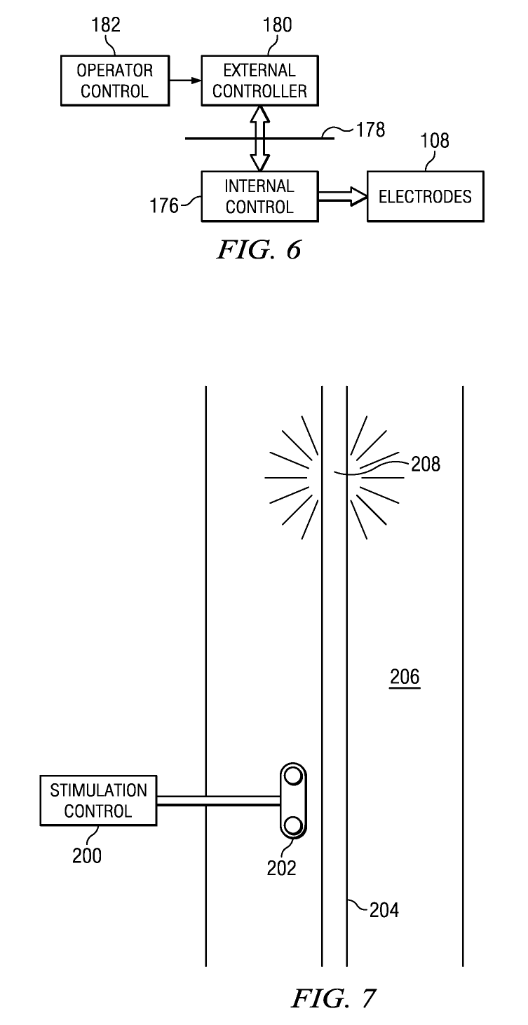

With reference to FIG. 6, a block diagram depicts a wireless neural stimulation system, in accordance with an embodiment. An operator control 182 communicates stimulation parameters to an external controller 180. The external controller 180 sends power and communication signals through a membrane 178, such as skin. An internal controller 176 receives the power and communication signals from the external controller 180. The internal controller 176 provides a stimulation pulse to electrodes 108.

With reference to FIG. 7, a diagram depicts a system for treating pain, in accordance with an embodiment. A nerve 204 within a limb or other body part 206 experiences a sensation of pain 208. For purposes of illustration, the neural processes are simplified. A stimulator 202 is placed proximate to the nerve 204, afferent to the pain signal 208. A stimulation control 200 provides a short-pulse stimulation to the stimulator 202, inducing parasthesia in the area of the stimulator 202. The induced parasthesia blocks the pain signals 208 from reaching the brain.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC Section 112 unless the exact words “means for” are followed by a participle.

The claims as filed are intended to be as comprehensive as possible, and NO subject matter is intentionally relinquished, dedicated, or abandoned.

What is claimed is:

1. A neural stimulation device comprising: an electrode; and a stimulus source connected to said electrode and delivering a stimulus to said electrode, wherein said stimulus has a voltage dissipation rate greater than 0.25V/μs.

2. The device of claim 1, wherein said stimulus has a leading edge rise rate greater than 0.25V/μs.

3. The device of claim 1, wherein said stimulus delivers a sub-threshold amount of charge to stimulate a nerve.

4. The device of claim 1, wherein said stimulus has an exponential waveform.

5. The device of claim 1, wherein said stimulus has a voltage dissipation rate greater than 0.50 V/μs.

6. The device of claim 1, wherein said stimulus has a voltage dissipation rate greater than 1.0 V/μs.

7. A method of stimulating nerves comprising: generating a stimulation voltage and delivering a stimulus wherein said stimulus has a voltage dissipation rate greater than 0.25V/μs.

8. The method of claim 7, wherein said stimulus has a leading edge rise rate greater than 0.25V/μs.

9. The method of claim 7, wherein said stimulus delivers a sub-threshold amount of charge to stimulate a nerve.

10. The method of claim 7, wherein said stimulus has an exponential waveform.

11. The method of claim 7, wherein said stimulus has a voltage dissipation rate greater than 0.50 V/μs.

12. The method of claim 7, wherein said stimulus has a voltage dissipation rate greater than 1.0 V/μs.

13. A nerve stimulation system comprising: a control device; a pulse generator communicably connected to said control device; and an electrode connected to said pulse generator, wherein said pulse generator receives a control signal from said control device and delivers a stimulation pulse to said electrodes, wherein said stimulation pulse has a voltage dissipation rate greater than 0.25V/μs

14. The system of claim 13, wherein said stimulation pulse has a leading edge rise rate greater than 0.25V/μs.

15. The system of claim 13, wherein said stimulation pulse delivers a sub-threshold amount of charge to stimulate a nerve.

16. The system of claim 13, wherein said stimulation pulse has an exponential waveform.

17. The system of claim 13, wherein said stimulus has a voltage dissipation rate greater than 0.50 V/μs.

18. The system of claim 13, wherein said stimulus has a voltage dissipation rate greater than 1.0 V/μs.

19. A method of neural stimulation comprising: generating a stimulation pulse delivering a charge quantity less than the charge injection stimulation threshold for a nerve; and providing said stimulation pulse to tissue proximate to said nerve.

20. The method of claim 19 wherein said stimulation pulse has an exponential waveform.

21. A neural stimulation device comprising: an stimulation pulse generator; and an electrode connected to said stimulation pulse generator, wherein said stimulation pulse generator delivers a stimulation pulse to said electrode, wherein said stimulation pulse delivers an amount of charge less than the charge-injection stimulation threshold for a targeted nerve.

22. The device of claim 21 wherein said stimulation pulse has an exponential waveform.

23. A neural stimulation system comprising: an external control; and an internal control communicably connected to said external control; wherein said internal control generates a stimulation pulse in response to a control signal received from said external control, wherein said stimulation pulse delivers an amount of charge less than the charge-injection threshold for a targeted nerve.

24. The system of claim 23 wherein said stimulation pulse has an exponential waveform.

25. A method of stimulating a nerve comprising: generating a voltage between a first electrode and a second electrode and delivering a stimulation pulse to a nerve, wherein said stimulation pulse has an exponential waveform.

26. The method of claim 25, wherein said exponential waveform has a duration less than 50 μs.

27. The method of claim 25, wherein said exponential waveform is formed by the discharge of a capacitor.

28. The method of claim 27, wherein said capacitor has a capacitance less than 20 nF.

29. The method of claim 25, wherein said first electrode has a surface area less than three square millimeters.

30. The method of claim 25, wherein said exponential waveform has a voltage dissipation rate greater than 0.25V/μs.

31. The method of claim 25, wherein said stimulation pulse delivers an amount of charge less than the charge-injection threshold.

32. A nerve stimulation device comprising a stimulation generator; and electrodes connected to said stimulation generator, wherein said stimulation generator delivers a stimulation pulse having an exponential waveform to said electrodes.

33. The method of claim 32, wherein said exponential waveform has a duration less than 50 μs.

34. The method of claim 32, wherein said exponential waveform is formed by the discharge of a capacitor.

35. The method of claim 34, wherein said capacitor has a capacitance less than 20 nF.

36. The method of claim 32, wherein said first electrode has a surface area less than three square millimeters.

37. The method of claim 32, wherein said exponential waveform has a voltage dissipation rate greater than 0.25V/μs.

38. The method of claim 32, wherein said stimulation pulse delivers an amount of charge less than the charge-injection threshold.

39. A nerve stimulation system comprising an external controller and an internal controller communicably connected to said external controller, wherein said external controller provides a control signal to said internal controller and said internal controller generates an exponential stimulation pulse in response to said control signal.

40. The method of claim 39, wherein said exponential waveform has a duration less than 50 μs.

41. The method of claim 39, wherein said exponential waveform is formed by the discharge of a capacitor.

42. The method of claim 41, wherein said capacitor has a capacitance less than 20 nF.

43. The method of claim 39, wherein said first electrode has a surface area less than three square millimeters.

44. The method of claim 39, wherein said exponential waveform has a voltage dissipation rate greater than 0.25V/μs.

45. The method of claim 39, wherein said stimulation pulse delivers an amount of charge less than the charge-injection threshold.

46. A method of nerve stimulation, comprising: positioning electrodes in tissue adjacent to a nerve; providing a stimulation pulse to said electrodes, wherein said stimulation pulse does not include frequency components lower than 6000 Hz.

47. A neural stimulator comprising: a power source; a pulse generator connected to said power source; and electrodes connected to said pulse generator; wherein said pulse generator provides a stimulation pulse to said electrodes, and said stimulation pulse does not include frequency components lower than 6000 Hz.

48. A neural stimulation system comprising: an external controller; an internal controller communicably connected to said external controller; and electrodes connected to said internal controller; wherein said internal controller provides a stimulation pulse to said electrodes, such that the stimulation pulse does not include frequency components lower than 6000 Hz.
